(19) United States
(12) Patent Application Publication
(10) Pub. No.: US 2011/0262569 A1
(43) Pub. Date: Oct. 27, 2011

(54) Complex and Manufacturing Method Therefor

(75) Inventors: Morihiro Aoyagi, Osaka (JP); Masaki Nakamura, Osaka (JP)

(73) Assignee: HOUSE FOODS CORPORATION, OSAKA (JP)

(21) Appl. No.: 13/141,632

(22) PCT Filed: Dec. 24, 2009
(86) PCT No.: PCT/JP2009/071472

§ 371 Date: Jun. 22, 2011

(30) Foreign Application Priority Data
Dec. 24, 2008 (JP) 2008-328261

(51) Int. Cl. A61K 36/67; A61K 36/906; A23L 1/28; A23L 1/303; A23L 1/30; A23G 3/00; A61K 36/81; A61K 36/9066

(52) U.S. Cl. 424/734; 424/760; 424/756; 426/73; 426/72; 426/658; 426/655; 426/61

(57) ABSTRACT

Disclosed are a starting material, and a manufacturing method therefor, which starting material contains a lipophilic component with flavors or odors typical of pungent components, such as capsaicin, and bitter components, such as turmeric extract, and which is able to effectively suppress the taste and/or odor of the lipophilic component, while also not causing the ingredients to separate. Also disclosed is a composition for food and drink, cosmetics, and medicine, etc., to which this starting material is added. Further disclosed is a liquid composition in a form in which said starting material is dispersed in water. Provided is a complex that contains a lipophilic component, a substance (A) selected from a group comprising plant sterols, γ-oryzanol, isoflavone, vitamin D, vitamin E, vitamin K, and combinations thereof, and cyclodextrin. Also provided is a composition to which the complex has been added. Further provided is a liquid composition containing the complex, water, and thickener in a form in which the complex is dispersed in water. Additionally provided is a manufacturing method for a complex that contains a lipophilic component, a substance (A) selected from a group comprising plant sterols, γ-oryzanol, isoflavone, vitamin D, vitamin E, vitamin K, and combinations thereof, and cyclodextrin, which complex manufacturing method comprises a complexing step, in which the lipophilic component, the substance (A), and cyclodextrin are mixed in the presence of water to form a complex.

### TECHNICAL FIELD

The present invention relates to a composite material in which a taste or an odor of a lipophilic component is inhibited, and/or decomposition/deterioration thereof is inhibited, and also to a manufacturing method therefor.

### BACKGROUND ART

As one of the lipophilic components having a taste or odor, Capsaicins, which are pungent components of capsicum pepper, for example, are known to have various effects useful for organisms, such as an effect of appetite stimulation, an effect of vasodilation and vasoconstriction, an effect of increasing salivation, an effect of increasing gastric acid secretion, an effect of increasing peristaltic movement of the intestinal tract, an effect of decreasing the cholesterol level in the circulatory system, an effect of increasing energy metabolism, and an effect of increasing the release of bioactive peptides. However, capsaicins have a strong pungency, and hence the application range thereof to foods and beverages has been limited.

To inhibit the pungency of capsaicins, novel glycoside capsaicinoids have been proposed which are obtained by modifying the molecular structure of capsaicinoids to eliminate their strong pungency (Patent Document 1). However, the glycoside capsaicinoids are novel chemically synthesized compounds, and hence are not approved for use in foods or beverages.

Meanwhile, a masking agent which is characterized by including a polyglycerin condensed ricinoleate and a food including the masking agent have been proposed (Patent Document 2). Specifically, a masking agent-containing chili oil has been disclosed which is obtained by adding 0.1% of a capsicum pepper extract oil and 0.5% of hexaglycerin condensed ricinoleate to sesame oil. However, the application range of this masking agent is limited to foods and beverages containing a large oil component, and in addition, this masking agent is likely to affect the flavor of foods and beverages because of a waxy odor thereof.

In addition, lipophilic components are decomposed due to interaction with water, or interaction with light, an enzyme, oxygen, heat, or the like in the presence of water. With relation to a method for inhibiting such decomposition, some food packaging materials have been proposed (Patent Document 3) in which the antimicrobial effects of isothiocyanate are retained even after heat drying in the following manner. Specifically, the stability of isothiocyanate is improved in such a manner that isothiocyanate included in a cyclodextrin is kneaded with a synthesis resin to form films, sheets and trays, or contained in a printing ink or a paint, which is then printed or applied onto films. These are stable in dry state, but cannot retain sufficient storage stability in a state where water content is high, for example, in beverages and high water content foods.

Meanwhile, a hydrophilic composite material of an L-ascorbic acid higher fatty acid ester imparted with stability with time, and stability against heat can be obtained by adding a fat-soluble L-ascorbic acid higher fatty acid ester to water or a hydrophilic solution in which a cyclodextrin is dissolved, and stirring the mixture at 50 to 100° C. (Patent Document 4). However, this method has a problem that especially substances unstable in the presence of water are likely to undergo reaction such as decomposition, because of contact with water or the hydrophilic solvent, and besides because of exposure to high-temperature during the inclusion. In addition, it cannot be said that the stability of the obtained composite material is sufficient.

### CITATION LIST

### Patent Documents

Patent Document 1: Japanese Patent No. 3156240
Patent Document 2: Japanese Patent Application Publication No. 2002-65177
Patent Document 3: Japanese Patent Application Publication No. Hei. 7-46973
Patent Document 4: Japanese Patent Application Publication No. Hei 10-231224

### SUMMARY OF THE INVENTION

### Problems to be Solved by the Invention

An object of the present invention is to provide a material comprising a lipophilic component having a taste and/or an odor typified by pungent components such as capsaicins, and bitter components such as turmeric extracts, and/or comprising a lipophilic component susceptible to decomposition/deterioration due to interaction with light, an enzyme, oxygen, heat, or the like, the material being capable of effectively inhibiting a taste and/or an odor of the lipophilic component, and/or capable of inhibiting decomposition/deterioration of the lipophilic component, as well as to provide a manufacturing method therefor.

In addition, another object of the present invention is to provide a composition, such as foods, beverages, cosmetics and pharmaceutical drugs, comprising the above-described material blended therein.

### Means for Solving the Problems

The present inventors have found that when a lipophilic component is composited with a phytosterol and a cyclodextrin, a taste or an odor of the lipophilic component can be inhibited. In addition, the present inventors also have found that a material obtained by compositing a lipophilic component with, among substances having a structure analogous to phytosterols, or the like, γ-oryzanol, isoflavone, vitamin D, vitamin E, or vitamin K being used instead of the phytosterol is capable of inhibiting a taste and an odor of the lipophilic component. These findings led to the completion of the present invention.

The present invention provides a composite material comprising: a lipophilic component; a substance (A) selected from the group consisting of phytosterols, γ-oryzanol, isoflavone, vitamin D, vitamin E, vitamin K, and combinations thereof; and a cyclodextrin.

The present invention also provides a composition comprising the composite material blended therein.

The present invention also provides a manufacturing method for a composite material comprising: a lipophilic component; a substance (A) selected from the group consisting of phytosterols, γ-oryzanol, isoflavone, vitamin D, vitamin E, vitamin K, and combinations thereof; and a cyclodextrin, the method comprising a compositing step of forming the composite material by mixing a lipophilic component; a substance (A); and a cyclodextrin in the presence of water.

### Effects of the Invention

The present invention can provide a material comprising a lipophilic component having a taste and/or an odor typified by pungent components such as capsaicins, and bitter components such as turmeric extracts, and/or a lipophilic component susceptible to decomposition/deterioration due to interaction with light, an enzyme, oxygen, heat, or the like, the material being capable of effectively inhibiting a taste and/or an odor of the lipophilic component, and/or capable of inhibiting decomposition/deterioration of the lipophilic component, as well as a manufacturing method therefor.

The present invention also makes it possible to provide compositions, such as foods and beverages, cosmetics, pharmaceutical drugs, each of which comprising such a material blended therein.

### BRIEF DESCRIPTION OF THE DRAWINGS

FIG. 1 is a graph showing a ratio of remaining capsaicin at an acylase concentration of (i) 0.7 unit/ml.

FIG. 2 is a graph showing a ratio of remaining capsaicin at an acylase concentration of (ii) 13 unit/ml.

FIG. 3 is a graph showing change in ratio of remaining capsinoids.

### MODES FOR CARRYING OUT THE INVENTION

A composite material of the present invention comprises: a lipophilic component; a substance (A) selected from the group consisting of phytosterols, γ-oryzanol, isoflavone, vitamin D, vitamin E, vitamin K, and combinations thereof; and a cyclodextrin.

Typical examples of the lipophilic component include capsaicins, which are one of lipophilic pungent components for example. The capsaicins include capsaicin, dihydrocapsaicin, nordihydrocapsaicin, homocapsaicin, vanillylnonanamide, and vanillyl butyl ether. Since they contain a large amount of capsaicins, capsicum pepper extracts such as capsicum oleoresins are suitably used as a raw material containing capsaicins.

Meanwhile, examples of the lipophilic component other than the capsaicins include: (6)-gingerol, (6)-shogaol, zingerone, and (8),(10)-shogaol, which are pungent components of ginger; piperine and piperanine, which are pungent components of pepper; sanshool, which is a pungent component Japanese pepper; and the like. A pepper extract, a ginger extract, and a Japanese pepper extract can be suitably used as a raw material containing pungent components of pepper, ginger, and Japanese pepper, respectively.

Besides the pungent components, the present invention can be applied to lipophilic bitter components such as a turmeric extract containing a lipophilic component with a bitter taste. Moreover, the present invention can be applied not only to the above-described lipophilic components of spices, but also to unsaturated fatty acids such as docosahexaenoic acid, and eicosapentaenoic acid.

In addition, the composite material of the present invention has found to be capable of inhibiting the decomposition of the lipophilic component, for example, due to interaction with water, or interaction with light, an enzyme, oxygen, heat, or the like in the presence of water. In other words, the composite material of the present invention stabilizes the lipophilic component, and improves the storability of the lipophilic component. Accordingly, for example, substances having a structure analogous to capsaicins but having no pungency, such as capsinoids, unsaturated fatty acids, curcumin, and the like can be suitably used as the lipophilic component. The composite material of the present invention is effective for improvement in stability of these substances.

The phytosterol used in the present invention is a cyclic higher alcohol which has a steroid skeleton having one or two double bonds, and which has a hydroxyl group at the C-3 position and a hydrocarbon side chain at the C-17 position, and is contained in a plant. Examples of common phytosterols include sitosterol, campesterol, stigmasterol, and the like, and any one of these can be used.

γ-Oryzanol used in the present invention is a composite compound obtained from the seed coat of Oryza sativa of the Poaceae family, and mainly formed by ester-bonding a triterpene alcohol to ferulic acid.

Any vitamins D, E, and K and isoflavone can be used in the present invention, as long as the vitamins D, E, and K and isoflavone are prepared for food or pharmaceutical drug application.

The cyclodextrin used in the present invention refers to a cyclic non-reducing maltooligosaccharide, whose constitutional unit is glucose. Even though any one of α-cyclodextrin with six glucose units, β-cyclodextrin with seven glucose units, and γ-cyclodextrin with eight glucose units may be used, γ-cyclodextrin is preferable since γ-cyclodextrin is decomposed by human digestive enzymes and since γ-cyclodextrin is easy to use for foods and beverages, particularly for beverages because of its high solubility in water.

The composite material of the present invention can be manufactured by a method comprising a compositing step of forming the composite material by mixing a lipophilic component; a substance (A) selected from the group consisting of phytosterols, γ-oryzanol, isoflavone, vitamin D, vitamin E, vitamin K, and mixtures thereof; and a cyclodextrin in the presence of water. For manufacturing the composite material of the present invention, the amount of the substance (A) is preferably 0.5 to 30000 parts by weight with respect to one part by weight of the lipophilic component. Meanwhile, the amount of the cyclodextrin is, for example, preferably 0.01 to 1000 parts by weight, and more preferably 0.1 to 100 parts by weight, with respect to one part by weight of the substance (A). Meanwhile, the amount of water coexisting in manufacturing the composite material is, for example, preferably 0.01 to 100 parts by weight, and more preferably 0.1 to 10 parts by weight, with respect to one part by weight of the cyclodextrin. In addition, when the composite material of the present invention is manufactured, the mixing is preferably conducted under heating at 40 to 90° C., more preferably 50 to 85° C.

In manufacturing the composite material, the order of adding or mixing water, the lipophilic component, the substance (A), and the cyclodextrin is not particularly limited. For example, the composite material can be formed as follows: the cyclodextrin and water are mixed together to prepare a mixture; then the substance (A) is added thereto, followed by mixing to homogeneity; and subsequently the lipophilic component is added thereto followed by mixing. However, the order is not limited thereto, and, for example, the lipophilic component, the substance (A), the cyclodextrin, and water may be mixed with each other simultaneously. Note that the mixing here is performed preferably by using a mixing apparatus with high shearing force, such as a kneader, for forming the composite material by thoroughly kneading these components.

The obtained composite material can be in any form. For example, by using a vehicle or the like, the composite material may be formed into a powder form or a granular form. The composite material may be in a liquid form or paste form where the composite material is dispersed or emulsified in a solvent such as water.

The thus obtained composite material of the present invention is advantageous in that the taste and/or odor of the lipophilic component is effectively inhibited. The inhibition of the taste and/or odor in the composite material of the present invention has a mechanism different from that of what is called masking involving addition of a sweet component, or the like. It is uncertain what structure the composite material of the present invention has; however, it is conceivable that at least the lipophilic component contained in the composite material of the present invention is in such a state that the lipophilic component cannot bind to the receptor of the taste.

In addition, the composite material of the present invention is capable of inhibiting the decomposition or deterioration of the lipophilic component, for example, due to interaction with water, or interaction with light, an enzyme, oxygen, heat, or the like in the presence of water. In other words, the composite material of the present invention can stabilize the lipophilic component, and improve the storability of the lipophilic component. The composite material of the present invention is easily dispersed in water. Hence, the composite material can be blended into foods or beverages, pharmaceutical drugs, cosmetics and the like, and provided as various compositions. More specifically, examples of the food or beverage into which the composite material of the present invention is blended include beverages, jellies, tablets, and the like. Here, a case where the composite material of the present invention is blended into a beverage is taken as an example. A heat-sterilized beverage in a container can be produced, for example, as follows. The composite material of the present invention is added to water. An acidulant is added thereto to adjust the pH to 4.0 or less, preferably 2.5 to 3.5. Raw materials such as a sweetener, a fruit juice, a flavor, a coloring agent, and vitamin C are added and mixed thereto. This mixture is heated to 65 to 100° C. for sterilization treatment. Then, this mixture is filled into a container, and sealed. Moreover, a jelly in a container can be produced by adding a gelatinizing agent to the above-mentioned raw materials.

The composition of the present invention comprising the composite material, water and a thickener can be provided as a liquid composition in which the composite material is in a water dispersed form. Specifically, although the composite material tends to be deposited in water, the inclusion of the thickener makes it possible to provide a liquid composition in which the composite material is dispersedly held in water. This liquid composition can also be provided as a liquid composition in a container, such as a beverage in a container. In this case, there are advantages in that the oil components do not separate in the container and hence the oil components do not attach onto the inner surface of the container.

Here, examples of the thickener include gellan gum, fermentation-derived cellulose, xanthan gum, gum arabic, tamarind gum, guar gum, locust bean gum, karaya gum, tara gum, agar, gelatin, pectin, soybean polysaccharides, CMC (carboxymethylcellulose), carrageenan, microcrystalline cellulose, propylene glycol alginate, and the like. Of these, fermentation-derived cellulose is preferably used, from the viewpoints that the composite material is dispersed uniformly in water, and that the composite material has a favorable texture when ingested orally.

The amount of the thickener is not particularly limited, as long as the composite material can be dispersed in water with the amount. For example, it is preferable that 0.01 to 1.0% by weight of the thickener be contained in the liquid composition.

### INDUSTRIAL APPLICABILITY

The composite material of the present invention comprising: a lipophilic component; a substance (A) selected from the group consisting of phytosterols, γ-oryzanol, isoflavone, vitamin D, vitamin E, vitamin K, and combinations thereof; and a cyclodextrin is a composite material in which a taste and/or an odor of the lipophilic component is inhibited, or a composite material in which decomposition/deterioration of the lipophilic component due to interaction with light, an enzyme, oxygen, heat, or the like is inhibited. Hence, the composite material of the present invention can be applied to foods, beverages, cosmetics, pharmaceutical drugs, and the like, to which a lipophilic component having a taste and/or an odor, or undergoing decomposition/deterioration has not been applicable so far. Hence, the composite material of the present invention makes it possible to provide foods, beverages, cosmetics, pharmaceutical drugs, and the like, which have various effects useful for organisms, such as an effect of appetite stimulation, an effect of vasodilation and vasoconstriction, an effect of increasing salivation, an effect of increasing gastric acid secretion, an effect of increasing peristaltic movement of the intestinal tract, an effect of decreasing the cholesterol level in the circulatory system, an effect of increasing energy metabolism, and an effect of increasing the release of bioactive peptides.

### EXAMPLES

### Example 1 and Comparative Example 1

Into 0.44 parts by weight of distilled water, 0.44 parts by weight of γ-cyclodextrin was dissolved by mixing (in hot water at 60° C.). In Example 1, 0.12 parts by weight of β-sitosterol was added to the aqueous solution of γ-cyclodextrin, and then the mixture was vigorously stirred to homogeneity, while being heated to 60° C. in hot water. Meanwhile, in Comparative Example 1, 0.12 parts of distilled water was added to the aqueous solution of γ-cyclodextrin, and the mixture was vigorously stirred to homogeneity, while being heated to 60° C. in hot water. Next, 0.0011 parts by weight of a capsicum pepper extract was added thereto, and the mixture was stirred to homogeneity, while being heated to 60° C. in hot water. Moreover, the total amount was adjusted to 100 parts by weight by adding distilled water thereto. The blending ratios (in part by weight) of Example 1 and Comparative Example 1 are shown in the following Table 1.

TABLE 1

| | | Comparative |
|---|---|---|
| Material name | Example 1 | Example 1 |
| γ-cyclodextrin | 0.44 | 0.44 |
| distilled water | 0.44 | 0.44 |
| β-sitosterol | 0.12 | — |
| distilled water | — | 0.12 |
| capsicum pepper extract | 0.0011 | 0.0011 |
| (capsicum oleoresin | | |
| (capsaicins content: 40 | | |
| weight percent)) | | |
| distilled water | 99.0 | 99.0 |
| total | 100.0 | 100.0 |

The pungency was more markedly inhibited in Example 1 than in Comparative Example 1.

### Example 2 and Comparative Example 2

Into 0.40 parts by weight of distilled water, 0.40 parts by weight of γ-cyclodextrin was dissolved by mixing (in hot water at 60° C.). In Example 2, 0.11 parts by weight of β-sitosterol was added to the aqueous solution of γ-cyclodextrin, and then the mixture was vigorously stirred to homogeneity, while being heated to 60° C. in hot water. Meanwhile, in Comparative Example 2, 0.11 parts of distilled water was added to the aqueous solution of γ-cyclodextrin, and the mixture was vigorously stirred to homogeneity, while being heated to 60° C. in hot water. Next, 0.10 parts by weight of a ginger extract was added thereto, and the mixture was stirred to homogeneity, while being heated to 60° C. in hot water. Moreover, the total amount was adjusted to 100 parts by weight by adding distilled water thereto. The blending ratios (in part by weight) of Example 2 and Comparative Example 2 are shown in the following Table 2.

TABLE 2

| | | Comparative |
|---|---|---|
| Material name | Example 2 | Example 2 |
| γ-cyclodextrin | 0.40 | 0.40 |
| distilled water | 0.40 | 0.40 |
| β-sitosterol | 0.11 | — |
| distilled water | — | 0.11 |
| ginger extract | 0.10 | 0.10 |
| (containing gingerol and | | |
| sanshool) | | |
| distilled water | 98.99 | 98.99 |
| total | 100.0 | 100.0 |

The pungency was more markedly inhibited in Example 2 than in Comparative Example 2.

### Example 3 and Comparative Example 3

Into 0.04 parts by weight of distilled water, 0.04 parts by weight of γ-cyclodextrin was dissolved by mixing (in hot water at 60° C.). In Example 3, 0.01 parts by weight of β-sitosterol was added to the aqueous solution of γ-cyclodextrin, and then the mixture was vigorously stirred to homogeneity, while being heated to 60° C. in hot water. Meanwhile, in Comparative Example 3, 0.01 parts of distilled water was added to the aqueous solution of γ-cyclodextrin, and the mixture was vigorously stirred to homogeneity, while being heated to 60° C. in hot water. Next, 0.014 parts by weight of a pepper extract was added thereto, and the mixture was stirred to homogeneity, while being heated to 60° C. in hot water. Moreover, the total amount was adjusted to 100 parts by weight by adding distilled water thereto. The blending ratios (in part by weight) of Example 3 and Comparative Example 3 are shown in the following Table 3.

TABLE 3

| | | Comparative |
|---|---|---|
| Material name | Example 3 | Example 3 |
| γ-cyclodextrin | 0.04 | 0.04 |
| distilled water | 0.04 | 0.04 |
| β-sitosterol | 0.01 | — |
| distilled water | — | 0.01 |
| pepper extract | 0.014 | 0.014 |
| (piperine content: 92 | | |
| weight percent or more) | | |
| distilled water | 99.9 | 99.9 |
| total | 100.0 | 100.0 |

The pungency was more inhibited in Example 3 than in Comparative Example 3.

### Example 4 and Comparative Example 4

Into 0.04 parts by weight of distilled water, 0.04 parts by weight of γ-cyclodextrin was dissolved by mixing (in hot water at 60° C.). In Example 4, 0.01 parts by weight of β-sitosterol was added to the aqueous solution of γ-cyclodextrin, and then the mixture was vigorously stirred to homogeneity, while being heated to 60° C. in hot water. Meanwhile, in Comparative Example 4, 0.01 parts of distilled water was added to the aqueous solution of γ-cyclodextrin, and the mixture was vigorously stirred to homogeneity, while being heated to 60° C. in hot water. Next, 0.016 parts by weight of a Japanese pepper extract was added thereto, and the mixture was stirred to homogeneity, while being heated to 60° C. in hot water. Moreover, the total amount was adjusted to 100 parts by weight by adding distilled water thereto. The blending ratios (in part by weight) of Example 4 and Comparative Example 4 are shown in the following Table 4.

TABLE 4

| | | Comparative |
|---|---|---|
| Material name | Example 4 | Example 4 |
| γ-cyclodextrin | 0.04 | 0.04 |
| distilled water | 0.04 | 0.04 |
| β-sitosterol | 0.01 | — |
| distilled water | — | 0.01 |
| Japanese pepper extract | 0.016 | 0.016 |
| (Japanese pepper oil | | |
| (containing sanshool)) | | |
| distilled water | 99.9 | 99.9 |
| total | 100.0 | 100.0 |

The pungency was more inhibited in Example 4 than in Comparative Example 4.

### Example 5 and Comparative Example 5

Into 0.04 parts by weight of distilled water, 0.04 parts by weight of γ-cyclodextrin was dissolved by mixing (in hot water at 60° C.). In Example 5, 0.01 parts by weight of β-sitosterol was added to the aqueous solution of γ-cyclodextrin, and then the mixture was vigorously stirred to homogeneity, while being heated to 60° C. in hot water. Meanwhile, in Comparative Example 5, 0.01 parts of distilled water was added to the aqueous solution of γ-cyclodextrin, and the mixture was vigorously stirred to homogeneity, while being heated to 60° C. in hot water. Next, 0.018 parts by weight of a turmeric extract was added thereto, and the mixture was stirred to homogeneity, while being heated to 60° C. in hot water. Moreover, the total amount was adjusted to 100 parts by weight by adding distilled water thereto. The blending ratios (in part by weight) of Example 5 and Comparative Example 5 are shown in the following Table 5.

TABLE 5

| | | Comparative |
|---|---|---|
| Material name | Example 5 | Example 5 |
| γ-cyclodextrin | 0.04 | 0.04 |
| distilled water | 0.04 | 0.04 |
| β-sitosterol | 0.01 | — |
| distilled water | — | 0.01 |
| turmeric extract | 0.018 | 0.018 |
| (turmeric oil (containing | | |
| essential oil constituent | | |
| with a bitter taste)) | | |
| distilled water | 99.9 | 99.9 |
| total | 100.0 | 100.0 |

The pungency was more inhibited in Example 5 than in Comparative Example 5.

Next, Examples 6 to 10 and Comparative Examples 6 and 7 show effects of inhibiting pungency in the cases where substances (γ-oryzanol, vitamin D, vitamin E, vitamin K, isoflavone, saponin, and catechin) having an analogous structure or the like to that of the phytosterol were used instead of the phytosterol for the compositing.

### Example 6

Into 0.79 parts by weight of distilled water, 0.79 parts by weight of γ-cyclodextrin was dissolved by mixing (in hot water at 60° C.). 0.001 parts by weight of a pepper extract was added to the aqueous solution of γ-cyclodextrin, and the mixture was stirred to homogeneity, while being heated to 60° C. in hot water. Next, 0.021 parts by weight of γ-oryzanol was added thereto, and the mixture was vigorously stirred to homogeneity, while being heated to 60° C. in hot water. Moreover, the total amount was adjusted to 100 parts by weight by adding distilled water thereto.

### Example 7

Example 7 was conducted in the same manner as Example 6, except that 0.21 parts by weight of vitamin D was added instead of 0.21 parts by weight of γ-oryzanol.

### Example 8

Example 8 was conducted in the same manner as Example 6, except that 0.21 parts by weight of vitamin E was added instead of 0.21 parts by weight of γ-oryzanol.

### Example 9

Example 9 was conducted in the same manner as Example 6, except that 0.21 parts by weight of vitamin K was added instead of 0.21 parts by weight of γ-oryzanol.

### Example 10

Example 10 was conducted in the same manner as Example 6, except that 0.21 parts by weight of isoflavone was added instead of 0.21 parts by weight of γ-oryzanol.

### Comparative Example 6

Comparative Example 6 was conducted in the same manner as Example 6, except that 0.21 parts by weight of saponin was added instead of 0.21 parts by weight of γ-oryzanol.

### Comparative Example 7

Comparative Example 7 was conducted in the same manner as Example 6, except that 0.21 parts by weight of catechin was added instead of 0.21 parts by weight of γ-oryzanol.

### Comparative Example 8

Comparative Example 8 was conducted in the same manner as Example 6, except that 0.21 parts by weight of distilled water was added instead of 0.21 parts by weight of γ-oryzanol.

The blending ratios (in part by weight) along with effects of inhibiting pungency of Examples 6 to 8 and Comparative Examples 6 to 8 are shown in the following Table 6. As shown in Table 6, pungency was inhibited in the composite material comprising γ-oryzanol, vitamin D, vitamin E, vitamin K, and isoflavone, whereas pungency was not inhibited in the composite material comprising saponin and catechin.

TABLE 6

| | | | | | | Comparative | Comparative | Comparative |
|---|---|---|---|---|---|---|---|---|
| Material name | Example 6 | Example 7 | Example 8 | Example 9 | Example 10 | Example 6 | Example 7 | Example 8 |
| γ-cyclodextrin | 0.79 | 0.79 | 0.79 | 0.79 | 0.79 | 0.79 | 0.79 | 0.79 |
| distilled water | 0.79 | 0.79 | 0.79 | 0.79 | 0.79 | 0.79 | 0.79 | 0.79 |
| capsicum pepper | 0.001 | 0.001 | 0.001 | 0.001 | 0.001 | 0.001 | 0.001 | 0.001 |
| extract (capsicum | | | | | | | | |
| oleoresin | | | | | | | | |
| (capsaicins content: | | | | | | | | |
| 40 weight percent)) | | | | | | | | |
| γ-oryzanol | 0.21 | — | — | — | — | — | — | — |
| (Oryza Oil & Fat | | | | | | | | |
| Chemical Co., Ltd.) | | | | | | | | |
| vitamin D (DSM | — | 0.21 | — | — | — | — | — | — |
| Corporate) | | | | | | | | |
| vitamin E (DSM | — | — | 0.21 | — | — | — | — | — |
| Corporate) | | | | | | | | |
| vitamin K (J-OIL | — | — | — | 0.21 | — | — | — | — |
| MILLS, INC.) | | | | | | | | |
| isoflavone (J-OIL | — | — | — | — | 0.21 | — | — | — |
| MILLS, INC.) | | | | | | | | |
| saponin (J-OIL | — | — | — | — | — | 0.21 | — | — |
| MILLS, INC.) | | | | | | | | |
| catechin | — | — | — | — | — | — | 0.21 | — |
| (DSM Corporate) | | | | | | | | |
| distilled water | — | — | — | — | — | — | — | 0.21 |
| distilled water | 98.99 | 98.99 | 98.99 | 98.99 | 98.99 | 98.99 | 98.99 | 98.99 |
| total | 100.0 | 100.0 | 100.0 | 100.0 | 100.0 | 100.0 | 100.0 | 100.0 |
| effect of inhibiting | ∘ | ∘ | ∘ | ∘ | ∘ | x | x | — |
| pungency | | | | | | | | |

∘ Pungency is inhibited compared with Comparative Example 8
x Pungency is not inhibited compared with Comparative Example 8

Next, Examples 11 and 12 and Comparative Example 9 show effects of inhibiting decomposition of a capsicum pepper extract due to interaction with an enzyme in the presence of water.

### Example 11

Into 1100 mg of distilled water, 1100 mg of γ-cyclodextrin was dissolved by mixing (in hot water at 60° C.). To the aqueous solution of γ-cyclodextrin, 2.8 mg of a capsicum pepper extract, and 300 mg of 13-sitosterol were added, and the mixture was vigorously stirred to homogeneity, while being heated to 60° C. in hot water.

### Comparative Example 12

Into 2200 mg of distilled water, 2200 mg of γ-cyclodextrin was dissolved by mixing (in hot water at 60° C.). To the aqueous solution of γ-cyclodextrin, 2.8 mg of a capsicum pepper extract, and 600 mg of 13-sitosterol were added, and the mixture was vigorously stirred to homogeneity, while being heated to 60° C. in hot water.

### Comparative Example 9

Into 1100 mg of distilled water, 1100 mg of γ-cyclodextrin was dissolved by mixing (in hot water at 60° C.). To the aqueous solution of γ-cyclodextrin, 2.8 mg of a capsicum pepper extract, and 300 mg of distilled water were added, and the mixture was vigorously stirred to homogeneity, while being heated to 60° C. in hot water.

Note that, blend amounts of Examples 11 and 12 and Comparative

Example 9 are shown in the following Table 7.

TABLE 7

| Raw material (in part | Example | | Comparative |
|---|---|---|---|
| by weight) | 11 | Example 12 | Example 9 |
| γ-cyclodextrin | 1100 mg | 2200 mg | 1100 mg |
| distilled water | 1100 mg | 2200 mg | 1100 mg |
| β-sitosterol | 300 mg | 600 mg | — |
| distilled water | — | — | 300 mg |
| capsicum pepper extract | 2.8 mg | 2.8 mg | 2.8 mg |
| (capsicum oleoresin | | | |
| (capsaicins content: 40 weight | | | |
| percent)) | | | |
| total | 2502.8 mg | 5002.8 mg | 2502.8 mg |

Into Falcon tubes (50 ml), 1.9 g, 3.8 g, and 1.9 g of the composite materials of Example 11, Example 12, and Comparative Example 9, respectively, prepared as described above were weighed, and each sample was diluted to 30 ml with a 50 mM Tris-HCl solution. Into a Falcon tube (50 ml), 0.9 ml of each of the samples thus diluted was measured, and 100 μl of an acylase was added. Here, the acylase was used in two different forms: Acylase (i) 0.7 unit/ml (in Examples 11 and 12, and Comparative Example 9), and Acylase (ii) 13 unit/ml (in Example 12 and Comparative Example 9), and enzymatic decomposition using each form was investigated. The sample was held in a constant temperature water bath shaker at 37° C. for 13 hours in the cases of acylase (i), and was held at 37° C. for 60 minutes therein in the cases of Acylase (ii). Then, after the above-described reaction time had elapsed, 1.5 ml of water, and 0.5 ml of a 2.5 N sodium hydroxide solution were added thereto, and a boiling treatment was conducted thereon for 10 minutes.

Thereafter, the sample was diluted with methanol to 12.5 ml, and then 0.5 ml of a 2.5 N hydrochloric acid solution was added thereto. Then, the mixture was again diluted with methanol to 25.0 ml. The thus prepared sample was filtered through a filter, and used as a sample for liquid chromatography measurement.

For liquid chromatography, a fluorescence detector was used, and the measurement was carried out under the following conditions.

Column: mightysil RP-18 GP Aqua 250-2.0 (5 μm); Flow Rate: 0.2 ml/min; Mobile Phase: 50% acetonitrile and 50% TFA-water (pH 3.3); Injection Amount: 10 μl.

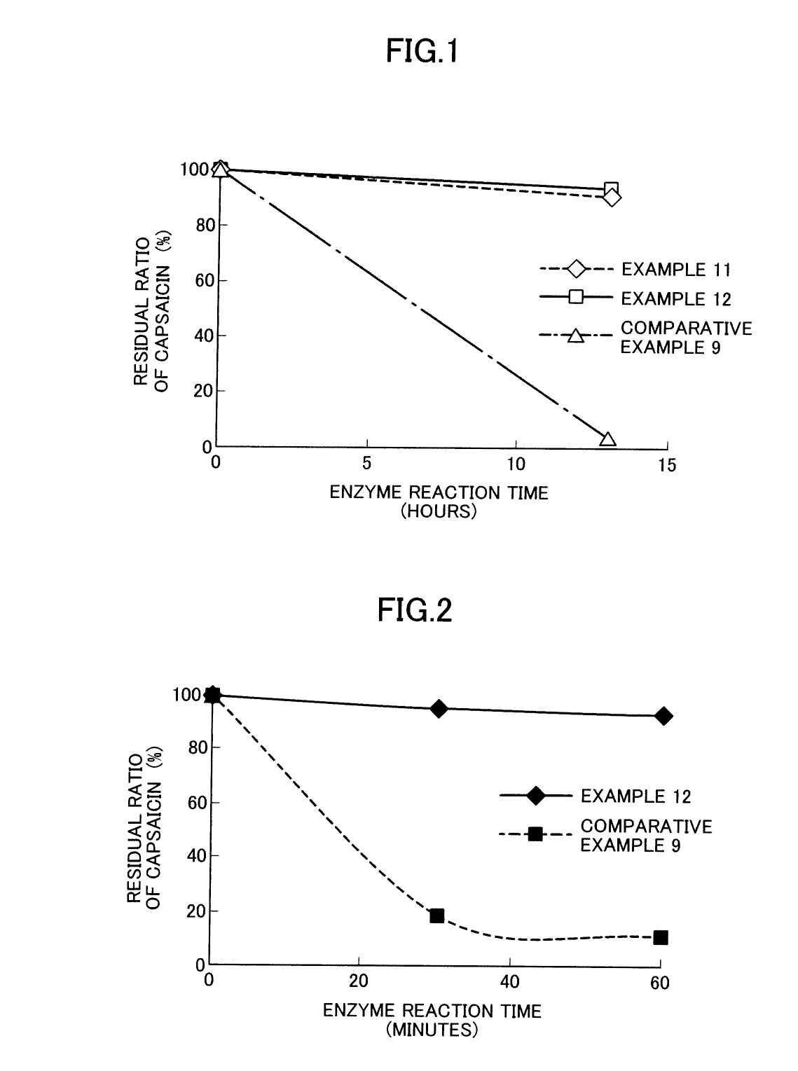

As shown in FIGS. 1 and 2, by forming composite materials using β-sitosterol and γ-cyclodextrin, enzymatic decomposition of capsaicin was successfully inhibited, and the stability of capsaicin was successfully improved.

### Example 13

As an unsaturated fatty acid, DHA (“DHA-22HG”, a deodorized fish oil containing 22% or more of DHA and manufactured by Maruha Nichiro Foods, inc.) was used.

Mixed preliminary were 0.44 parts by weight of γ-cyclodextrin and 0.12 parts by weight of β-sitosterol. The mixture of γ-cyclodextrin and β-sitosterol was added to 0.44 parts by weight of water heated to 80° C., and the mixture was stirred with a mixer. Moreover, 0.50 parts by weight of DHA heated to 80° C. was added, and the mixture was stirred with a mixer to obtain a composite material. Into 97.75 parts by weight of water, 1.5 parts by weight of the obtained composite material, 0.50 parts by weight of citric acid, and 0.25 parts by weight of trisodium citrate were dispersed, and the mixture was stirred with a mixer for 30 seconds. Thus, a model beverage containing composite material was prepared.

### Example 14

As an unsaturated fatty acid, DHA (“DHA-22HG”, a deodorized fish oil containing 22% or more of DHA and manufactured by Maruha Nichiro Foods, inc.) was used.

Mixed preliminary were 0.44 parts by weight of γ-cyclodextrin and 0.12 parts by weight of β-sitosterol. The mixture of γ-cyclodextrin and β-sitosterol was added to 0.44 parts by weight of water heated to 80° C., and the mixture was stirred with a mixer. Moreover, 0.05 parts by weight of DHA heated to 80° C. was added, and the mixture was stirred with a mixer to obtain a composite material. Into 98.20 parts by weight of water, 1.05 parts by weight of the obtained composite material, 0.50 parts by weight of citric acid, and 0.25 parts by weight of trisodium citrate were dispersed, and the mixture was stirred with a mixer for 30 seconds. Thus, a model beverage containing composite material was prepared.

### Comparative Example 10

As an unsaturated fatty acid, DHA (“DHA-22HG”, a deodorized fish oil containing 22% or more of DHA and manufactured by Maruha Nichiro Foods, inc.) was used.

0.44 parts by weight of γ-cyclodextrin was added to 0.56 parts by weight of water heated to 80° C., and the mixture was stirred with a mixer. Moreover, 0.50 parts by weight of DHA heated to 80° C. was added, and the mixture was stirred with a mixer to obtain a composite material. Into 97.75 parts by weight of water, 1.5 parts by weight of the obtained composite material, 0.50 parts by weight of citric acid, and 0.25 parts by weight of trisodium citrate were dispersed, and the mixture was stirred with a mixer for 30 seconds. Thus, a model beverage containing composite material was prepared.

### Comparative Example 11

As an unsaturated fatty acid, DHA (“DHA-22HG”, a deodorized fish oil containing 22% or more of DHA and manufactured by Maruha Nichiro Foods, inc.) was used.

0.44 parts by weight of γ-cyclodextrin was added to 0.56 parts by weight of water heated to 80° C., and the mixture was stirred with a mixer. Moreover, 0.05 parts by weight of DHA heated to 80° C. was added, and the mixture was stirred with a mixer to obtain a composite material. Into 98.20 parts by weight of water, 1.05 parts by weight of the obtained composite material, 0.50 parts by weight of citric acid, and 0.25 parts by weight of trisodium citrate were dispersed, and the mixture was stirred with a mixer for 30 seconds. Thus, a model beverage containing composite material was prepared.

The model beverages containing composite material prepared as described above were stored at room temperature for two days, and subjected to sensory evaluation where the intensity of a distinctive oxidation odor generated upon oxidation of DHA was represented in three levels.

From the results shown in the following Table 8, it can be seen that when the present invention was employed, the distinctive oxidation odor of DHA was successfully reduced. In other words, the present invention successfully inhibited the oxidation of the unsaturated fatty acid, and improved the stability of the component.

TABLE 8

| Raw material (in | Example | Comparative | Example | Comparative |
|---|---|---|---|---|
| part by weight) | 13 | Example 10 | 14 | Example 11 |
| deodorized fish oil | 0.50 | 0.50 | 0.05 | 0.05 |
| containing DHA | | | | |
| (“DHA-22HG” | | | | |
| manufactured by | | | | |
| Maruha Nichiro | | | | |
| Foods, inc.) | | | | |
| β-sitosterol | 0.12 | — | 0.12 | — |
| γ-cyclodextrin | 0.44 | 0.44 | 0.44 | 0.44 |
| water | 0.44 | 0.56 | 0.44 | 0.56 |
| citric acid | 0.50 | 0.50 | 0.50 | 0.50 |
| trisodium citrate | 0.25 | 0.25 | 0.25 | 0.25 |
| water | 97.75 | 97.75 | 98.20 | 98.20 |
| total | 100 | 100 | 100 | 100 |
| result of sensory | ++ | +++ | + | ++ |
| evaluation | | | | |

Evaluation Criteria
+: No oxidation odor was noticed.
++: Slight oxidation odor was noticed.
+++: Strong oxidation odor was noticed.

### Example 15

Capsinoids extracted from “Natura” manufactured by AJINOMOTO CO., INC., were used.

Into a mortar, 3.5 parts by weight of water heated to 70° C., 0.70 parts by weight of β-sitosterol, and 0.35 parts by weight of a fat and fatty oil containing the capsinoids were placed, and kneaded. Moreover, 7.0 parts by weight of γ-cyclodextrin was added thereto, and the mixture was kneaded in a hot water bath at 70° C. for 10 minutes. Thus, a composite material was prepared. Into 87.6 parts by weight of water, 11.55 parts by weight of the obtained composite material, 0.56 parts by weight of citric acid, and 0.27 parts by weight of trisodium citrate were dispersed, and the dispersion was stirred with a mixer for 30 seconds. Thus, a model beverage containing composite material was prepared. The model beverage containing composite material was heated up to 93° C., and sterilized by being held at 90° C. for 3 minutes, and then filled into a pouch. Thereafter, the pouch was held in a constant-temperature water bath at 83° C. for 7 minutes to perform second sterilization.

### Comparative Example 12

Capsinoids extracted from “Natura” manufactured by AJINOMOTO CO., INC., were used.

Into a mortar, 10.5 parts by weight of water heated to 70° C., 0.70 parts by weight of β-sitosterol, and 0.35 parts by weight of a fat and fatty oil containing the capsinoids were placed, and kneaded. To 87.3 parts by weight of water heated to 70° C., 0.33 parts by weight of an emulsifier (a polyglycerin fatty acid ester SWA-10D manufactured by Mitsubishi-Kagaku Foods Corporation) and 11.55 parts by weight of the kneaded product of β-sitosterol and the fat and fatty oil containing the capsinoids were added, and the mixture was stirred with a mixer for 3 minutes. Moreover, 0.56 parts by weight of citric acid, and 0.27 parts by weight of trisodium citrate were added, and the mixture was stirred with a mixer for 30 seconds. Thus, an emulsion-containing model beverage was prepared. The emulsion-containing model beverage was heated up to 93° C., and sterilized by being held at 90° C. for 3 minutes, and then filled into a pouch. Thereafter, the pouch was held in a constant-temperature water bath at 83° C. for 7 minutes to perform second sterilization.

### Comparative Example 13

Capsinoids extracted from “Natura” manufactured by AJINOMOTO CO., INC., were used.

To 0.70 parts by weight of refined rapeseed oil heated to 70° C., 0.35 parts by weight of a fat and fatty oil containing the capsinoids was added, and dissolved thereinto. On the other hand, 7.0 parts by weight of γ-cyclodextrin, and 3.5 parts by weight of water were introduced into a mortar, and mixed with each other in a hot water bath at 70° C. to obtain a paste. To this paste, 1.05 parts by weight of the above-described oil phase in which the capsinoids were dissolved was added, and the mixture was kneaded in a hot water bath at 70° C. for 10 minutes. Thus, a composite material was prepared. Into 87.6 parts by weight of water, 11.55 parts by weight of the obtained composite material, 0.56 parts by weight of citric acid, and 0.27 parts by weight of trisodium citrate were dispersed, and the dispersion was stirred with a mixer for 30 seconds. Thus, a model beverage containing composite material was prepared. The model beverage containing composite material was heated up to 93° C., and sterilized by being held at 90° C. for 3 minutes, and then filled into a pouch. Thereafter, the pouch was held in a constant-temperature water bath at 83° C. for 7 minutes to perform second sterilization.

TABLE 9

| | Example | Comparative | Comparative |
|---|---|---|---|
| Raw material (in part by weight) | 15 | Example 12 | Example 13 |
| fat and fatty oil containing | 0.35 | 0.35 | 0.35 |
| capsinoids | | | |
| (extracted from “Natura” | | | |
| manufactured by AJINOMOTO | | | |
| CO., INC.) | | | |
| β-sitosterol | 0.70 | 0.70 | — |
| refined rapeseed oil | — | — | 0.70 |
| (manufactured by J-OIL MILLS, | | | |
| INC.) | | | |
| γ-cyclodextrin | 7.0 | — | 7.0 |
| water | 3.5 | 10.5 | 3.5 |
| emulsifier | — | 0.33 | — |
| (SWA-10D manufactured by | | | |
| Mitsubishi-Kagaku Foods | | | |
| Corporation) | | | |
| citric acid | 0.56 | 0.56 | 0.56 |
| trisodium citrate | 0.27 | 0.27 | 0.27 |
| water | 87.6 | 87.3 | 87.6 |
| total | 100 | 100 | 100 |

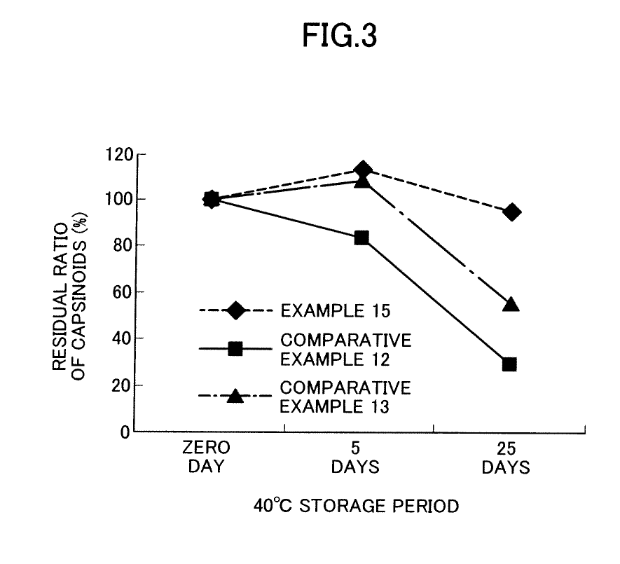

The model beverages prepared in Example 15, and Comparative Examples 12 and 13 were stored at 40° C. After certain periods of time had elapsed, the capsinoids in the samples were quantitatively determined by liquid chromatography. For the ratio of remaining capsinoids, values determined after the beverages were stored for 5 days and for 25 days at 40° C. were represented by percentage, with a value of the capsinoids immediately after the start of the storage (zero days) being employed as 100%. FIG. 3 shows the results. As is apparent from FIG. 3, the decomposition of the capsinoids in the storage at 40° C. was more markedly inhibited in Example 15 than Comparative Examples 12 and 13. From the results described above, it has been found that the present invention makes it possible to inhibit the decomposition of capsinoids in the presence of water, and to improve the stability thereof.

### Pretreatment Method for Liquid Chromatography

Regarding Example 15, 12.5 g of the model beverage was centrifuged (at 3000 rpm for 10 minutes), and then the supernatant was removed. To the deposit, 6 ml of DMSO (dimethyl sulfoxide) was added, and the mixture was ultrasonicated to dissolve the deposit. Moreover, the mixture was diluted with methanol to 25 ml, filtered through a 0.45-μm filter, and then used as a test liquid.

Regarding each of Comparative Examples 12 and 13, 5 g of the model beverage was sampled, and diluted with methanol to 10 ml, filtered through a 0.45-μm filter, and then used as a test liquid.

### Measurement Conditions for Liquid Chromatography

A fluorescence detector was used.

| | |
|---|---|
| Column | mightysil (250 mm, φ 2.0) |
| Flow rate | 0.2 ml/min |
| Injection Amount | 3 μl |
| Mobile Phase | pH 3.3 TFA-water:acetonitrile = 20:80 |
| FLD Detector | EX270 EM330 |

1. A composite material comprising:
a lipophilic component;
a substance (A) selected from the group consisting of phytosterols, γ-oryzanol, isoflavone, vitamin D, vitamin E, vitamin K, and combinations thereof; and
a cyclodextrin.

2. The composite material according to claim 1, manufactured by a method comprising a compositing step of forming the composite material by mixing the lipophilic component, the substance (A), and the cyclodextrin in the presence of water.

3. The composite material according to claim 1, wherein the lipophilic component is a pungent component or a bitter component.

4. The composite material according to claim 1, wherein the lipophilic component is a capsicum pepper extract, a ginger extract, a pepper extract, a Japanese pepper extract, or a turmeric extract.

5. The composite material according to claim 1, wherein the lipophilic component is a component susceptible to decomposition or deterioration due to interaction with light, an enzyme, oxygen, heat, or the like.

6. A composition comprising the composite material according claim 1 blended therein.

7. A manufacturing method for a composite material comprising:
a lipophilic component;
a substance (A) selected from the group consisting of phytosterols, γ-oryzanol, isoflavone, vitamin D, vitamin E, vitamin K, and combinations thereof; and
a cyclodextrin,
the method comprising:
a compositing step of forming the composite material by mixing the lipophilic component, the substance (A), and the cyclodextrin in the presence of water.

8. The composite material according to claim 2, wherein the lipophilic component is a pungent component or a bitter component.

9. The composite material according to claim 2, wherein the lipophilic component is a capsicum pepper extract, a ginger extract, a pepper extract, a Japanese pepper extract, or a turmeric extract.

10. The composite material according to claim 2, wherein the lipophilic component is a component susceptible to decomposition or deterioration due to interaction with light, an enzyme, oxygen, heat, or the like.

11. A composition comprising the composite material according claim 2 blended therein.
